(19) United States
(12) Patent Application Publication
(10) Pub. No.: US 2019/0083170 A1
(43) Pub. Date: Mar. 21, 2019

(54) SURGICAL INSTRUMENT, IN PARTICULAR ELECTROSURGICAL INSTRUMENT

(71) Applicant: OLYMPUS WINTER & IBE GMBH, Hamburg (DE)

(72) Inventors: Carlos GROTH, Berlin (DE); Christopher SPRENGER, Berlin (DE)

(73) Assignee: OLYMPUS WINTER & IBE GMBH, Hamburg (DE)

(21) Appl. No.: 16/192,031

(22) Filed: Nov. 15, 2018

Related U.S. Application Data
(63) Continuation of application No. PCT/EP2017/060629, filed on May 4, 2017.

(30) Foreign Application Priority Data
May 18, 2016 (DE) 10 2016 208 541.7

(51) Int. Cl. A61B 18/14; A61B 90/98

(52) U.S. Cl. CPC A61B 18/1477; A61B 2018/00988; A61B 90/98

(57) ABSTRACT

The disclosed embodiments relate to a surgical instrument, in particular an electrosurgical instrument. The surgical instrument is configured with a FRAM storage device for individually recognizing or detecting the surgical instrument. The disclosed embodiments further relate to the use of a FRAM storage device.

### CROSS REFERENCE TO RELATED APPLICATIONS

This application is a continuation of PCT/EP2017/060629 filed on May 4, 2017, which in turn claims priority to DE 10 2016 208 541.7 filed on May 18, 2016, the contents of which are incorporated by reference herein in their entirety.

### BACKGROUND

The disclosed embodiments relate to a surgical instrument, in particular an electrosurgical instrument. The disclosed embodiments further relate to the use of a Ferroelectric Random-Access Memory (FRAM) storage device.

It is known that electrosurgical instruments are used, for example, during the electrosurgical coagulation and/or ablation of biological tissue. In the process, electrodes of the instrument are supplied with a high-frequency voltage (HF voltage) of different potential (bipolar), so that the tissue surrounding the electrodes is warmed up to such an extent that the body's own proteins denature.

In addition, it is known that a high-frequency alternating current is conducted through tissue that is to be treated surgically, in order to deliberately damage or section said tissue during high-frequency surgery (also called HF surgery). In the case of this operating technique, hemostasis takes place at the same time as the sectioning due to the occlusion or respectively atrophying of the relevant vessels. During high-frequency surgery, an electrode, which is positioned on a surgical instrument, for example on a resectoscope, is deployed instead of a scalpel.

In many cases, an electrosurgical system comprises a HF generator having a rating of several 100 Watts and an operating voltage of 1000 Volts and more. A HF signal produced by the HF generator is applied to the HF electrode. During monopolar HF surgery, one connector of the high-voltage output is connected to the HF electrode. The second connector is brought into contact with a large patient electrode. Starting from the HF electrode, the current used for the treatment flows through the patient back to this patient electrode. During bipolar HF surgery, the HF electrode used for the treatment comprises two poles, between which the current produced by the HF generator flows. During high-voltage discharge, a plasma forms at the HF electrode, which produces the medical treatment, that is to say the cutting action and/or coagulation.

Furthermore, it is known that surgical instruments are equipped with so-called RFID (Radio Frequency Identification) tags, as a result of which a clear identification of the surgical instrument is guaranteed, such that information that is typical of the instrument is saved in a RFID chip. The RFID chip is read or respectively written to in a wired or contactless manner.

### SUMMARY

The object of the disclosed embodiments includes improving the handling of surgical instruments, in particular electrosurgical instruments.

This object is achieved by a surgical instrument, in particular an electrosurgical instrument, having a FRAM storage device for individually recognizing or detecting the surgical instrument.

The fact that non-volatile electronic memory types, based on FRAM technology, are used for the storage device of a RFID tag of the surgical instrument means that it is possible to reliably identify the surgical instruments and, for example, to document the treatment and/or manufacturing history thereof. In this case, e.g. the RFID tag of the surgical instrument is equipped with a FRAM memory, which makes it possible, for example, to sterilize surgical instruments which have been used as single-use instruments up to now by means of gamma radiation such that, following sterilization by means of the gamma radiation, the available information stored in the FRAM memory is still available and is not deleted, and the sterilized instrument can be reused. Up to now, bipolar applicators have been used for high-frequency surgery, for example as single-use instruments, which are now configured with a FRAM memory according to the disclosed embodiments, as a result of which the available information in the memory and, if applicable, the instrument can continue to be usable following sterilization by means of the gamma radiation.

This makes it possible to recognize the used instrument constantly, securely and automatically. In this case, it is in particular possible to recognize the sterilized instrument following sterilization by means of gamma radiation and to activate certain surgical instruments for surgical use. In addition, it is likewise possible, in the case of surgical instruments which are used as single-use instruments, to prevent the instrument configured with a FRAM memory being reused after the sterilization has been performed, since the single-use instrument is detected or respectively recognized following the gamma radiation sterilization, and is no longer reused following recognition of the information and data stored in the FRAM memory.

In an exemplary embodiment, the FRAM storage device is arranged or mounted in a shaft or in a housing of the surgical instrument, e.g. a handle.

Moreover, in exemplary embodiments, the surgical instrument has a cable, in particular a power cable or a connecting cable, wherein the FRAM storage device is arranged or provided in or on the cable or wherein the cable is configured with a FRAM storage device. In particular, the cable is permanently connected to the surgical instrument and thus, clearly identifies the surgical instrument.

Moreover, in an exemplary embodiment instrument, the cable of the surgical instrument is configured with a connecting coupling, in particular a plug, wherein the connecting coupling is configured with the FRAM storage device. In this case, the FRAM storage device is arranged or provided in or on the connecting coupling. In particular, a plug configured as a connecting coupling has a FRAM storage device which is preferably arranged in the interior of the plug or respectively in the housing of the plug.

Moreover, the surgical instrument is preferably configured as a, preferably bipolar, applicator for electrosurgically coagulating and/or for ablating biological tissue. In this case, the bipolar applicators configured as hand-held instruments are used in high-frequency (HF) surgery or radiofrequency (RF) surgery. Within the framework of the disclosed embodiments, it is further possible that a monopolar high-frequency (HF) applicator or ultrasonic applicator or microwave applicator has a FRAM storage device.

The object is further achieved by the use of a FRAM storage device with a surgical instrument or in a surgical instrument, in particular an electrosurgical instrument, which has been described above. To avoid repetitions, reference is expressly made to the above embodiments.

Further features of the disclosed embodiments will become evident from the description of embodiments according to the disclosed embodiments together with the claims and the appended drawing. The disclosed embodiments can fulfill individual features or be combined with one or more other different embodiments.

### BRIEF DESCRIPTION OF THE DRAWINGS

The disclosed embodiments will be described below by means of an exemplary embodiment with reference to the drawings. Reference is expressly made to the drawings regarding all of the details according to the disclosed embodiments which are not explained in greater detail in the text. The figures show the following:

FIG. 1 schematically shows an electrosurgical instrument, and

FIGS. 2a and 2b schematically show a plug of an electrosurgical instrument in a top view (FIG. 2a) and in a cross-section (FIG. 2b).

### DETAILED DESCRIPTION OF EMBODIMENTS

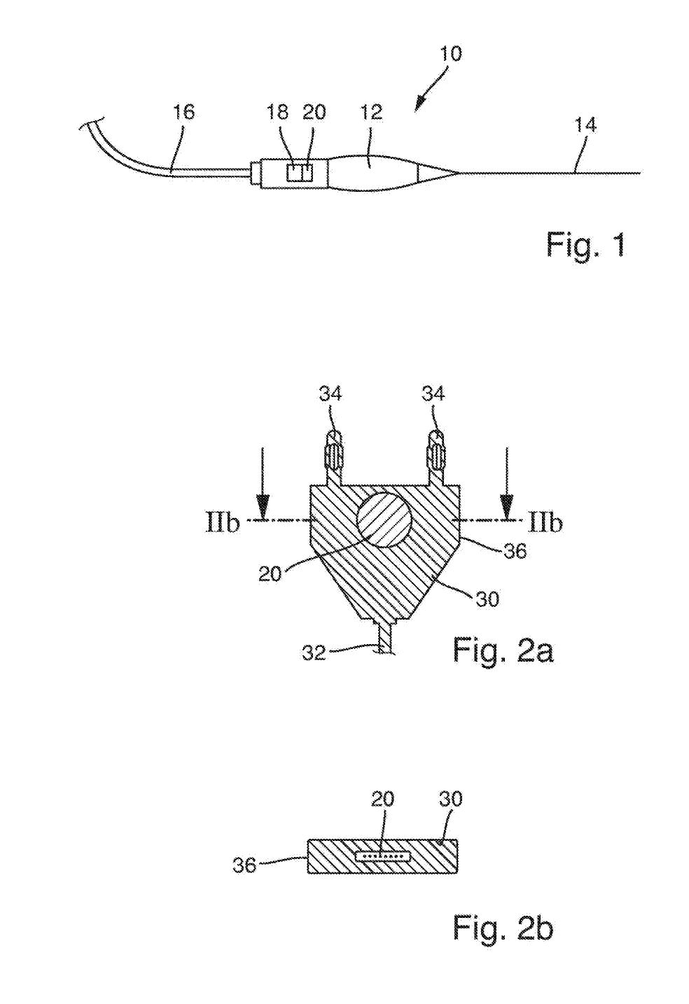

An electrosurgical instrument 10 is shown schematically in FIG. 1. The electrosurgical instrument 10 is preferably configured here as a bipolar electrosurgical instrument.

The electrosurgical instrument 10 comprises a handle 12 in the form of a shaft, at one end of which an applicator needle 14 is arranged. The applicator needle 14 is configured at the tip with two coagulation electrodes which are electrically separated from one another by an insulator. It is possible to section and coagulate biological tissue by means of the coagulation electrodes.

In order to operate the electrosurgical instrument 10, the surgical instrument 10 is connected to a supply unit by means of a wire 16 at the end of the handle 12 facing away from the applicator needle 14. The coagulation electrodes are supplied with a voltage by means of the wire 16 when a surgical instrument 10 is connected.

In addition, the handle 12 can have appropriate switches or respectively activation switches in order to operate the surgical instrument 10.

According to the disclosed embodiments, it is provided that a RFID tag 18 which is configured with a FRAM memory 20 which is schematically drawn is arranged in the handle 12. It is possible to store individual characteristics of the surgical instrument 10 by means of the FRAM memory 20, and to read said characteristics, if required, by means of a reader. This makes it possible to recognize the surgical instrument 10 securely and automatically. In particular, the use of the FRAM memory 20 makes it possible to sterilize the surgical instrument 10 by means of gamma radiation, without data being deleted from the FRAM memory 20 during or respectively following the sterilization.

In an exemplary embodiment, it is possible for the RFID tag 18 having the FRAM memory 20 to be arranged on the wire 16, in order to successfully identify the surgical instrument 10.

A top view of an attachment plug 30 of an electrosurgical instrument (not represented here) is shown schematically in FIG. 2a. FIG. 2b schematically shows a cross-section through the plug 30 in accordance with the dashed wire IIb-IIb in FIG. 2a.

A high-frequency unit (for electrosurgery) is connected, for example, to a surgical instrument by means of the attachment plug 30. To this end, the attachment plug 30 is connected to a connection cable 32, e.g. a HF connection cable, in order to conduct current, in particular HF current, from a high-frequency unit to the electrosurgical instrument. The attachment plug 30, which is configured as a connecting coupling, has two plug-in pins 34 as contacts or contact pins at its plug-in side, which plug-in pins are or can be plugged into e.g. a plug receptacle of a high-frequency unit or the like. Moreover, further details have been dispensed with better representing the disclosed embodiments.

The attachment plug 30 has a, preferably insulating, housing 36, in which a FRAM memory 20 is arranged or accommodated. In the interior of the housing 36, the FRAM memory 20 is surrounded by a cladding material of the plug 30. It is possible to reliably identify the surgical instrument equipped with and connected to the attachment plug 30 and to document, for example, the treatment and/or manufacturing history thereof by means of the data and information stored in the FRAM memory 20.

The attachment plug 30 shown in FIGS. 2a and 2b with the FRAM memory 20 is configured as a flat plug. Within the framework of the disclosed embodiments, the attachment plug 30 can also have only one pin 34 or more than two plug-in pins, i.e. at least one or more plug-in pins. The attachment plug 30 can further be configured with a different shape to the flat plug shape

The disclosed embodiments may be performed by individual features or a combination of multiple features disclosed herein. Features which are labeled with “in particular” or “preferably” are to be understood to be optional features.

1. A surgical instrument comprising:
a FRAM storage device for individually recognizing or detecting the surgical instrument.

2. The surgical instrument according to claim 1, wherein the FRAM storage device is arranged in a shaft of the surgical instrument.

3. The surgical instrument according to claim 1, wherein the surgical instrument has a cable, wherein the FRAM storage device is arranged in the cable.

4. The surgical instrument according to claim 3, wherein the cable of the surgical instrument is configured with a connecting coupling that is configured with the FRAM storage device.

5-6. (canceled)

7. The surgical instrument according to claim 4, wherein the connecting coupling is a plug.

8. The surgical instrument according to claim 1, wherein the surgical instrument is configured as an applicator for electrosurgically coagulating or for ablating biological tissue.

9. The surgical instrument according to claim 8, wherein the applicator is bipolar.

10. The surgical instrument according to claim 1, wherein the FRAM storage device is arranged in a housing of the surgical instrument.

11. The surgical instrument according to claim 1, wherein the FRAM storage device is mounted on a shaft of the surgical instrument.

12. The surgical instrument according to claim 1, wherein the FRAM storage device is mounted on a housing of the surgical instrument.

13. The surgical instrument according to claim 3, wherein the cable is at least one of a power cable or a connecting cable.

14. The surgical instrument according to claim 1, wherein the surgical instrument has a cable, wherein the FRAM storage device is mounted on the cable.
